(19) United States
(12) Patent Application Publication
(10) Pub. No.: US 2003/0060427 A1
(43) Pub. Date: Mar. 27, 2003

(54) Local injection prescription

(75) Inventors: Mitsukazu Matsumoto, Chiryu-shi (JP); Hisatake Ishikawa, Nagoya-shi (JP)

Correspondence Address: BEYER WEAVER & THOMAS LLP, P.O. BOX 778, BERKELEY, CA, 94704-0778, US

(73) Assignee: Mitsukazu Matsumoto

(21) Appl. No.: 09/954,513

(22) Filed: Sep. 10, 2001

(51) Int. Cl. A61K 31/7048

(52) U.S. Cl. 514/28

(57) ABSTRACT

A local injection prescription that disperses a malignant tumor to decrease the size of the tumor or terminate the tumor. The local injection prescription is produced by dissolving an organic compound having a lactone nucleus in lower alcohol and water.

### BACKGROUND OF THE ART

The present invention relates to a prescription prepared for local injections to treat malignant tumors that are commonly seen in mammals.

The general approach for treating a malignant tumor is to incise the lesion or administer an anti-tumor preparation. However, the incision of the lesion to treat cancer may result in contraction of the portion surrounding the lesion and formation of a cicatrix. In such case, the portion surrounding the lesion does not heal properly and form the same healthy tissues as those prior to the incision. The administration of an anti-tumor preparation is performed to basically prevent the enlargement and spreading of the tumor. In this case, it is difficult for the anti-tumor preparation to act directly on the tumor and decrease the size of the tumor or terminate the tumor.

### SUMMARY OF THE INVENTION

Accordingly, it is an object of the present invention to provide a local injection prescription that disperses a malignant tumor to decrease the size of the tumor or terminate the tumor by directly injecting the prescription to the lesion.

To achieve the above object, the present invention provides a local injection prescription obtained by dissolving an organic compound, which has a lactone nucleus, in lower alcohol and water. The organic compound contains macrolide. Macrolide includes avermectin.

Further, the organic compound includes phenytoin.

The lower alcohol is an alcohol selected from a group of alcohols represented by the molecular formulas of CH₄O, C₂H₆O, C₃H₈O, and C₄H₁₀O.

Lactone is a substance defined as an anhydride of hydroxy acid. Further, lactone is dehydrated and condensed to form the lactone nucleus. Macrolide is one example of an organic compound having the lactone nucleus. Macrolide is the generic term for substances that have a frame using a large lactone nucleus as a chemical structure. Further, macrolide is often included in antibiotics obtained from actinomycetes. Avermectin is one type of macrolide. Natural avermectin is obtained by fermenting actinomycete streptomyces avermitilis. Ivermectin is a widely known avermectin and is 2,2,2,3-dihydroavermectin B1.

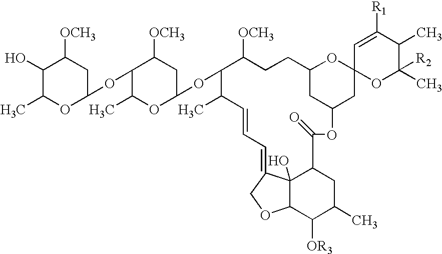

TABLE 1

| | R₁ | R₂ | R₃ |
|---|---|---|---|
| A1a | H | C₂H₅ | CH₃ |
| A1b | H | CH₃ | CH₃ |
| A2a | OH | C₂H₅ | CH₃ |
| A2b | OH | CH₃ | CH₃ |
| B1a | H | C₂H₅ | H |
| B1b | H | CH₃ | H |
| B2a | OH | C₂H₅ | H |
| B2b | OH | CH₃ | H |

The chemical name of phenytoin is 5,5-diphenyl-2,4-imidazolidinedione and the chemical formula is C15H₁₂N₂₀. The structural formula of phenytoin is as follows:

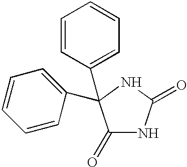

Table 2

The administration of macrolide as a substance having the lactone nucleus has shown that macrolide has an anti-tumor feature (Japanese Patent Publication Nos. 7-504913 and 7-504914). However, these observations have not focused on local usage. Although, it may be presumed that the local usage of macrolide as a substance having the lactone nucleus is effective, there have been no prescriptions for macrolide. It is possible to solely use a substance having the lactone nucleus. It is also possible to combine and use two or more substances having the lactone nucleus.

It is preferred that the lower alcohol be monatomic alcohol. Diatomic and triatmoic have high viscosity under normal temperatures and can thus not be solely used. It is further preferred that the monatomic alcohol be one of C₁ to C₄. It is especially preferred that the lower alcohol be methyl alcohol (CH₄O) and ethyl alcohol (C₂H₆O), which have small molecular weights. Although methyl alcohol may be used, the most preferred lower alcohol is ethyl alcohol since the amount used for local injections is small. Since the molecular weight of lower alcohol is small, lower alcohol easily passes through cell membranes. Further, since alcohol has a hydrophilic group and a hydrophobic group, alcohol may be used as a solvent that easily dissolves a substance having the lactone nucleus such as ivermectin. Additionally, alcohol easily permeates portions of cells having a high water content. In addition, ethanol is administered to treat hepar cancer. This is because alcohol inactivates glycogen, which is the energy source for growing cancer cells. Accordingly, alcohol is optimal for use as a prescription in the present invention.

However, the usage of ethyl alcohol having a high purity is painful when the ethyl alcohol permeates cells. It is thus preferred that the ratio of ethyl alcohol be 35 to 70 weight by percent. Once the alcohol permeates cells, pain is eliminated due to the peripheral nervous system blocking effect.

The prescription according to the present invention is used to treat malignant tumors that are commonly seen in mammals. Malignant tumors include sarcoma, adenoma, skin cancer, and the like. Further, mammals include humans.

### DETAILED DESCRIPTION OF THE PREFERRED EMBODIMENT

A preferred embodiment according to the present invention will now be discussed. In the preferred embodiment, IVOMEC (registered trademark of Merial Ltd.) injection, which is used as an anti-parasite preparation, was used as an ivermectin preparation. The preparation includes 10 mg of ivermectin in 1 ml.

ALEVIATIN (registered trademark of Dainippon Pharmaceutical Co., Ltd.) injection liquid, which was used as an anticonvulsant, was used as a phenytoin preparation. The preparation includes 50 mg of phenytoin in 1 ml.

Further, 70 percent ethyl alcohol was used as a solvent.

The solubility of IVOMEC injection and ALEVIATIN injection liquid relative to water and 70 percent ethyl alcohol solution was as shown in the following table.

TABLE 3

| | Aleviatin | Ethyl | | |
|---|---|---|---|---|
| IVOMEC | Injection | Alcohol | | |
| Injection | Liquid | Solution | Water | Dissolubility |
| 1 | | 1 | | |
| | 1 | 1 | | |
| 1 | 1 | 1 | | |
| 1 | | | 1 | |
| | 1 | | 1 | ∘ |
| 1 | 1 | 1 | | |
| 1 | 1 | 1 | 1 | |
| 1 | 1 | | | Δ |

Dissolved
1 indicates that 1 cc was added and agitated.
∘ Mostly Dissolved
The Solution was then left in a quiet state.
Turbid
Δ Viscous

Three types of prescriptions were used in the embodiment.

| | |
|---|---|
| Prescription 1 | |
| IVOMEC injection | 33.33 volume percent |
| 70 percent ethyl alcohol solution | 66.66 volume percent |
| Prescription 2 | |
| ALEVIATIN injection liguid | 33.33 volume percent |
| 70 percent ethyl alcohol solution | 66.66 volume percent |
| Prescription 3 | |
| IVOMEC injection | 22.22 volume percent |
| ALEVIATIN injection liquid | 11.11 volume percent |
| 70 percent ethyl alcohol solution | 66.66 volume percent |

In the following examples, the prescriptions were all directly injected to the lesion. However, the prescriptions may also be injected to the artery that supplies the lesion with blood.

### EXAMPLES

No. 1

Prescription 1 was locally injected to treat anus tumor in a large-size dog (male, weighing about 17 kg). An amount of 1.0 cc per injection was administered once a month for a total of six times. The observations were as shown in Table 4.

No. 2

Prescription 1 was locally injected to treat mastadenoma and melanoma in a medium-size dog (female, weighing about 13 kg). An amount of 1.0 cc per injection was administered once a month for a total of three times to the mastadenoma. An amount of 1.0 cc per injection was administered once to the melanoma. The observations were as shown in Table 4.

No. 3

Prescription 1 was locally injected to treat vaginal sarcoma in a medium-size dog (female, weighing about 10.5 kg). A total of 0.9 cc, 0.3 cc for each of three locations in the sarcoma, was injected and administered once a month for a total of two times. The observations were as shown in Table 4.

No. 4

Prescription 1 was locally injected to treat mastocytoma formed near the groin at the right rear leg in a large-size dog (male, weighing about 22 kg). An amount of 1.0 cc per injection was administered once a month for a total of two times. The observations were as shown in Table 4.

No. 5

Prescription 1 was locally injected to treat malignant skin histoma formed near the outer side of the left hip in a small-size dog (female, weighing about 4 kg). An amount of 0.2 cc per injection was administered once a month for a total of two times. The observations were as shown in Table 4.

No. 6

Prescription 1 was locally injected to treat subcutaneous lymphoma formed in the back of a small-size dog (female, weighing about 3 kg). An amount of 0.2 cc was injected and administered once a month for a total of two times. The observations were as shown in Table 4.

No. 7

Prescription 2 was locally injected to treat mastadenoma formed in the left and right mammary glands of a small-size dog (male, weighing about 5 kg). Since tumors were confirmed at ten locations, instead of administering all of the prescription at one location, the prescription was injected at three locations for each administration. The injections were administered sequentially in a cyclic manner to cover every location. A total of 0.6 cc, 0.2 cc per tumor, was administered once a month for a total of ten times. The observations were as shown in Table 4.

No. 8

Prescription 1 was locally injected to treat the right mammary gland for mastadenoma in a large-size dog (female, weighing about 20 kg). The prescription was injected at three locations of the sarcoma. An amount of 0.5 cc was injected at each of the three locations totaling to 1.5 cc per administration once a month for a total of two times. The observations were as shown in Table 4.

No. 9

Prescription 3 was locally injected to treat multiple skin cancer and chondrosarcoma of the entire body and the 16th rib in a large-size dog (male, weighing about 18 kg). An amount of 1.0 cc per injection was administered once a month for a total of two times to treat the multiple skin cancer. An amount of 1.0 cc per injection was administered once a month for a total of three times to treat the chondrosarcoma. The observations were as shown in Table 4.

No. 10

Prescription 1 was locally injected to treat osteosarcoma in the rear left femur of a medium-size dog (male, weighing about 15 kg). An amount of 1.0 cc per injection was administered once. The observations were as shown in Table 4.

No. 11

Prescription 1 was locally injected to treat folliculus pili cellular tumor in the neck and left back-side hip of a large-size dog. An amount of 0.5 cc was injected to the sarcoma at one location and an amount of 0.7 cc was injected to the sarcoma at another location. A total of 1.5 cc was administered once a month for a total of two times. The observations were as shown in Table 4.

No. 12

Prescription 1 was locally injected to treat upper jaw sinus sarcoma of the nasal cavity upper jaw sinus in a cat (male, weighing about 3 kg). An amount of 0.5 cc per injection was administered once a month for a total of three times. The observations were as shown in Table 4.

No. 13

Prescription 1 was locally injected to treat lymphoma of the rear right groin in a small-size dog (male, weighing about 5 kg) once. The observations were as shown in Table 4.

TABLE 4

| | Observation Results |
|---|---|
| 1 | Completely healed |
| 2 | Both completely healed |
| 3 | Completely healed |
| 4 | Completely healed |
| 5 | Completely healed |
| 6 | Completely healed |
| 7 | Reduced to 1/10 in tenth month |
| 8 | Reduced to ⅔ in first month |
| 9 | Both completely healed |
| 10 | Completely healed |
| 11 | Completely healed |
| 12 | Reduced to ½ in fourth month |
| 13 | Reduced to ½ in first month |

What is claimed is:

1. A local injection prescription produced by dissolving an organic compound having a lactone nucleus in lower alcohol and water.

2. The local injection prescription according to claim 1, wherein the organic compound is macrolide.

3. The local injection prescription according to claim 2, wherein the macrolide is avermectin.

4. The local injection prescription according to claim 1, wherein the organic compound is phenytoin.

5. The local injection prescription according to claim 1, wherein the lower alcohol is one selected from a group of alcohols represented by the molecular formulas of CH₄O, C₂H₆O, C₃H₈O, and C₄H₁₀O.

6. The local injection prescription according to claim 2, wherein the lower alcohol is one selected from a group of alcohols represented by the molecular formulas of CH₄O, C₂H₆O, C₃H₈O, and C₄H₁₀O.

7. The local injection prescription according to claim 3, wherein the lower alcohol is one selected from a group of alcohols represented by the molecular formulas of CH₄O, C₂H₆O, C₃H₈O, and C₄H₁₀O.

8. The local injection prescription according to claim 4, wherein the lower alcohol is one selected from a group of alcohols represented by the molecular formulas of CH₄O, C₂H₆O, C₃H₈O, and C₄H₁₀O.
